(19) United States
(12) Patent Application Publication
(10) Pub. No.: US 2025/0152181 A1
(43) Pub. Date: May 15, 2025

(54) REVISION GOAL POST OSTEOTOME

(71) Applicant: Zimmer, Inc., Warsaw, IN (US)

(72) Inventor: Julia Carlquist, Fort Wayne, IN (US)

(21) Appl. No.: 18/939,195

(22) Filed: Nov. 6, 2024

Related U.S. Application Data
(60) Provisional application No. 63/597,890, filed on Nov. 10, 2023.

(51) Int. Cl. A61B 17/15; A61B 17/14

(52) U.S. Cl. CPC A61B 17/151; A61B 17/142

(57) ABSTRACT

A surgical instrument, and method for use, where the surgical instrument is configured to be inserted into a bone. The surgical instrument can include an elongate arm, a handle having a force application surface, and a working end. The working end can include a first blade, a second blade facing the first blade, and a slot extending longitudinally between the first blade and the second blade.

### CLAIM OF PRIORITY

This application claims the benefit of U.S. Provisional Patent Application Ser. No. 63/597,890, filed on Nov. 10, 2023, the benefit of priority of which is claimed hereby, and which is incorporated by reference herein in its entirety.

### TECHNICAL FIELD

Examples described in this document pertain generally, but not by way of limitation, to surgical implements, devices and methods of use. More specifically, examples herein generally relate to surgical implements used in implant surgeries and revision surgeries and associated methods of use.

### BACKGROUND

Devices are regularly implanted or inserted in the human body. Such devices include implants for stabilizing bones, repairing joints, replacing biological systems with artificial components or the like. Each device implanted is a foreign object to the body. Further, each device implanted is usually not formed with biological material similar to the area of the human body in which it is inserted. In some examples, metal, plastic or other similar materials are implanted into the body, such as in a bone.

At times, an implanted device needs to be removed from the location in human body into which it was implanted. For example, an implanted device may need to be removed after a period of time from implantation within tissue, such as a bone. Some implants are multicomponent with one or more component inserted into the bone and other components coupled with the component implanted into the bone. In such an implant, the component implanted into the bone requires specific tools or implements to remove the component implanted from the bone. Sometimes, the component implanted into the bone benefits from a set of tools or implements that are complementary and each tool or implement has a specific function during a revision or removal surgery.

### SUMMARY

The present inventors have recognized, among other things, that a problem to be solved can include reducing the amount of time of surgical procedures or efforts exerted during a surgical procedure, such as revision procedures. In revision procedures, a reduction of time can occur by minimizing the number of surgical steps and providing tools that allow for a more efficient removal process. The present subject matter can help provide a solution to this problem, such as by providing a surgical tool with different faces, ends, points or other features that are beneficial when the surgical tool can be inserted into a body, such as around or proximate to an already existing implant. For example, a surgical instrument can have opposing faces, sides, ends or other similar configurations that can be inserted into specific locations in a body. For example, the surgical instrument can have a profile that can be placed on opposing sides of an implant. In an example, the medical professional can insert the surgical tool once to contact or be positioned around two separate locations in concert.

In examples, a surgical instrument can be configured to be inserted into a bone. The surgical instrument can include, for example, at least an elongate arm with a proximal end, a central body, and a distal end. The surgical implement can also include a handle coupled to the proximal end of an elongate arm having a force application surface, the handle coupled to the proximal end of the elongate arm and a working end proximate to the distal end of the elongate arm. In one example, the working end can include at least a first blade with an outer surface and an inwardly facing first surface and a second blade having an outer surface and an inwardly facing second surface. For instance, the first surface can face the second surface. In an example, the elongate arm can continuously taper from the central body to a distal end of each of the first blade and the second blade. The distal end of each of the first blade and the second blade can be configured to be inserted into the bone. The working end can also include, for example, a slot extending longitudinally between the first surface and the second surface. For example, the slot can be open at a distal end and can terminate in a closed configuration a specified distance away from the distal end.

In examples, a method of inserting a surgical instrument can be performed with a surgical instrument having a proximal end, a central body, a distal end, a first blade with a first inwardly facing surface extending distally from the central body, and a second, opposing blade with a second inwardly facing surface extending distally from the central body. The method can include aligning a distal end of the first blade and a distal end of the second blade on opposing sides of an object implanted into a bone. The example method can also include applying a lateral force to the proximal end of the surgical instrument where the force can be transferred from the proximal end of the surgical instrument to the distal end of the first blade and the distal end of the second blade. The example method can also include inserting into the bone the distal end of the first blade in concert with the distal end of the second blade, on opposing sides of the object with each application of the repeated force and ceasing the application of the force when the first blade and the second blade extend a specified distance into the bone. The example method can also include concurrently withdrawing the first blade and second blade from the bone.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an isometric view of a surgical implement in accordance with at least one example of the present disclosure.

FIG. 2 is a side, close up, view of a working end of a surgical implement in accordance with at least one example of the present disclosure.

FIG. 3 is a side, close up, view of a distal tip of a surgical implement in accordance with at least one example of the present disclosure.

FIG. 4 is a side view of a working end of a surgical implement in accordance with at least one example of the present disclosure.

FIG. 5 is a side view of a proximal end including a handle of a surgical implement in accordance with at least one example of the present disclosure.

FIG. 6A is a cross sectional view of an implanted object in accordance with at least one example of the present disclosure.

FIG. 6B is a top view of a surgical implement and an implanted object in accordance with at least one example of the present disclosure.

### DETAILED DESCRIPTION

An example of a surgical instrument used in surgical involving implanting or removing a medical device from a human body can include osteotomes. In some examples, an implanted medical device (an “implant” or an “implanted object”) can be inserted into, coupled with, or otherwise joined with, a tissue such as a bone. After inserting or coupling an implant with or into, a bone, additional tissue, such as new bone, can form around, along, in, or proximate to the implanted medical device. The new bone can form with the implant in a manner that the new bone and the implant are structurally joined or bonded together. After a period of time has passed, it can become difficult to separate the new bone from the implant.

In some situations, removal of an implant from a body can be desired or needed. A revision surgery, or surgery to remove or repair an implant, can be performed to remove at least part of the implant from the body. Revision surgeries can utilize specific surgical implements to release the implant from the body. In some examples, a bone can be coupled with an implanted medical device and in some situations, that same implanted medical device, should be or needs to be removed. For example, between the time of insertion of a medical device and removal, tissue, such as bone, can form on or around the implant. In such situations, separating the bone and/or tissue from the implant can require certain surgical implements. In some examples, surgical instruments can be designed specifically for removal of the implant. In other examples, generic surgical instruments can be useful to remove the implant.

Surgical instruments used for cutting or separating bone can include osteotomes. Osteotomes can be tailored for a specific surgery or can be generic to any surgery to remove an implant from a body. In one example, separating bone from an implant can be simplified by using a specific osteotome designed for separating bone from a specific implant. In another example, an osteotome can be compatible with an array of implants, such as a family of implants having different sizes.

An example of a method for removing an implant from a body can include inserting an osteotome into the area of the bone surrounding the perimeter of the implant. For example, a surgeon, or other medical professional, can repeatedly move the osteotome from one location to another location around an implant where the implant has been united with the bone. In some examples, a continuous connection between the bone and the surfaces of the implant has occurred. The osteotome can be inserted into the bone thereby breaking the connections formed between the bone and the implant. This can be a repetitive process and at times can be performed in areas of the bone with limited space between adjacent parts of the implant. For example, an implant that needs to be removed can have at least one part protruding or extending away from a central area of an implant which can pose as an obstacle when removing the implant. For instance, an osteotome should fit in and around the perimeter of the implant, including between small space around a perimeter of an implant. In some examples, the osteotome can be designed to have a profile that can fit within at least one part of an implant that protrudes or extends away from a central or other common area of the implant. This process can be time consuming with and can necessitate repetitive actions. The inventors have recognize altering the design of an osteotome can make revision surgeries more efficient.

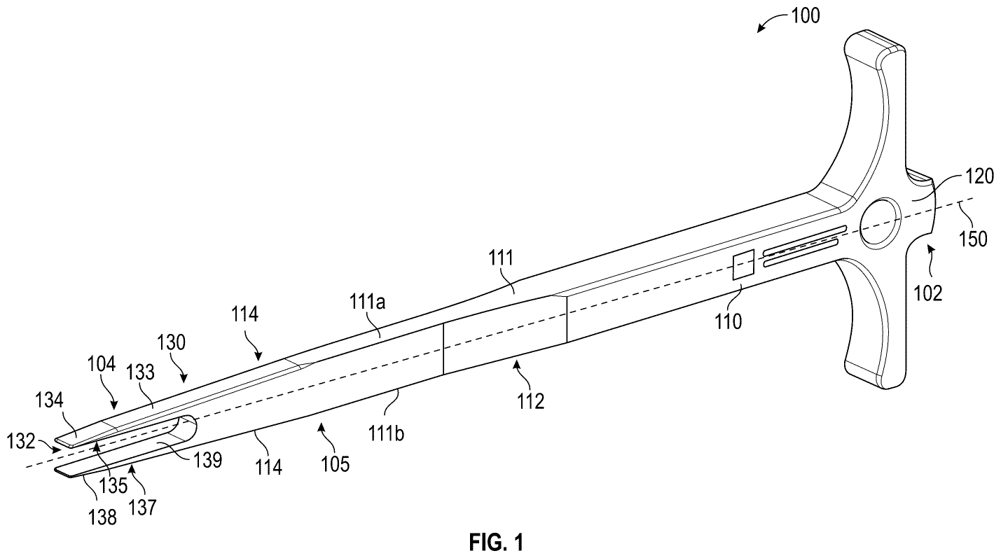

FIG. 1 illustrates a design of an osteotome 100 that can minimize the number of times an osteotome is inserted into a bone around an implant. In some examples, the osteotome 100 can reduce the frequency of tasks performed in a revision procedure making the revision procedure more efficient.

In an example, the osteotome 100 can include a proximal end 102 and a distal end 104. Interconnecting the proximal end 102 and the distal end 104 can be a central body 112. The proximal end 102 can include a handle 120. The distal end 104 can include a working end 130. The central body 112 can include an elongate arm 110 extending between and connecting proximal end 102 and the distal end 104. In an example, an elongate arm 110 can extend longitudinally from the handle 120 towards the distal end 104 interconnecting the handle 120 with the working end 130.

In an example, the working end 130 can include a pair of opposing blades 132. For example, a first blade 134 and a second blade 138 can each extend from a distal portion 105 of the central body 112. The first blade 134 and second blade 138 each can extend distally from the distal portion 105 of the central body 112 to the distal end 104 of the osteotome 100. In an example, the first blade 134 can be positioned on an opposing of a longitudinal axis 150 of the osteotome 100 from the second blade 138. For instance, the each of first blade 134 and the second blade 138 extend from the elongate arm 110 on opposing sides of the longitudinal axis 150. A first inner surface 135 of the first blade 134, for example, faces a second inner surface 139 of the second blade 138. In one example, the first inner surface 135 and the second inner surface 139 can be spaced about the longitudinal axis 150. For example, the longitudinal axis 150 can be approximately equidistantly spaced laterally between the first inner surface 135 and the second inner surface 139. In another example, the longitudinal axis 150 can be closer, laterally, to one of the first inner surface 135 or the second inner surface 139. A first blade outer surface 133 and a second blade outer surface 137 each can be a continuous surface extending from an outer surface 111 of the central body 112. For example, each of the first blade outer surface 133 and the second blade outer surface 137 extend continuously from a respective portion of the elongate arm 110.

The central body 112 can have a first lateral side 111a as the portion of the central body 112 positioned on the same side of the osteotome 100 as the first blade 134 and a second lateral side 111b positioned on the same side of the osteotome 100 as the second blade 138. For instance, the longitudinal axis 150 can extend between the first lateral side 111a and the second lateral side 111b. In an example, at least one of the first lateral side 111a and the second lateral side 111b can have a taper 114 extending from the distal portion 105 of the central body 112 to the distal end 104. The taper 114 can continue along each of the first lateral side 111a and the second lateral side 111b of the central body 112 towards the distal end 104. For example, the taper 114 can extend continuously from the distal portion 105 of the central body 112 and along of each of the first blade outer surface 133 and the second blade outer surface 137. In an example, the taper 114 can be continuous with the first blade outer surface 133 and the second blade outer surface 137 tapering towards each other.

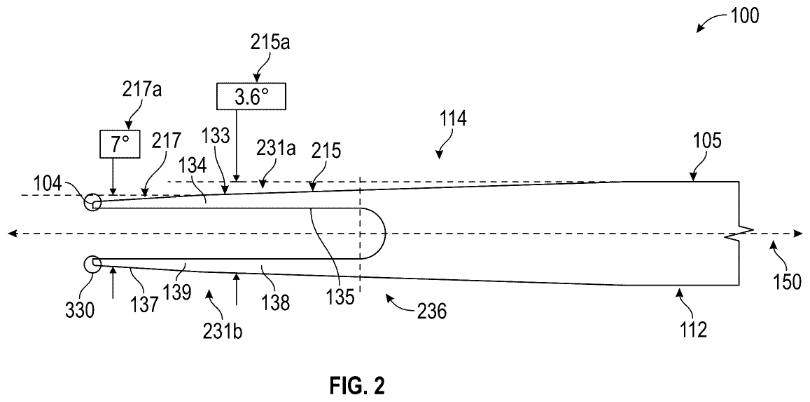

FIG. 2 illustrates a close-up view of the distal portion 105 of the central body 112 and the first blade 134 and the second blade 138. For example, FIG. 2 illustrates a close-up view of an example of the taper 114 that can extend along the first blade outer surface 133 and the second blade outer surface 137. In the example in FIG. 2, the taper 114 can begin along an outer surface of the central body 112. The taper 114, in one example, can begin at or proximate to a distal portion 105 of the central body 112. For instance, the taper 114 can begin at or proximate to the distal portion 105 and can be continuously angled towards a longitudinal axis 150 and the distal end 104 of the osteotome 100.

In an example, at least one of the first blade 134 and the second blade 138 can have a tapered outer surface. The outer surface of the central body 112 can also have a tapered profile that tapers towards at least one of the first blade 134 and the second blade 138. The first blade outer surface 133 and the second blade outer surface 137 each can taper toward the longitudinal axis 150. In one example, there can be a first taper 215 extending from the distal portion 105 of the central body 112 to a central portion 231a of the first blade 134. In another example, there can be a first taper 215 from the distal portion 105 of the central body 112 to a central portion 231b of the second blade 138. Optionally, there can be a second taper 217 extending from the central portion 231b of the second blade to the distal end 104 of at least one of the first blade 134 or second blade 138. The second taper 217, in one example, can have a greater taper angle 217a than the first taper angle 215a. For example, the first taper 215 can extend at a first angle 215a from the distal portion 105 to the central portion 231a and the second taper 217 can have a greater angle extending continuously from the first taper 215. The second taper 217 can extend at the greater angle from the first taper 215 to the distal end 104. For example, the first taper 215 can have an angle between about 1 and about 7 degrees relative to the longitudinal axis 150. In an example, the first taper 215 can have an angle between about 2 and about 4 degrees relative to the longitudinal axis 150. In another example, the first taper 215 can be 3.6 degrees. The second taper 217 can extend from the termination of the first taper 215 to the distal end 104 and can have an angle between about 5 and about 10 degrees relative to the longitudinal axis 150. In another example, the second taper 217 can have an angle between about 5 and about 8 degrees relative to the longitudinal axis. In yet another example, the second taper 217 can have an angle of about 7 degrees relative to the longitudinal axis 150. The angle of the first taper 215 and the second taper 217 can be any specified angle that can allow for ease in insertion into a tissue, such as a bone. In an example, the area where the first inner surface 135 meets a distal portion of the second taper 217 and the second inner surface 139 meets an opposing second taper 217 can form a point that can be sharp enough to be inserted into a tissue, such as a bone.

In an example, the first inner surface 135 of the first blade 134 and the second inner surface 139 of the second blade 138 can be substantially planar and in some instances can be parallel or substantially parallel relative to the longitudinal axis 150, such that the first inner surface 135 and the second inner surface 139 can face each other. In the example with the first inner surface 135 and the second inner surface 139 parallel or substantially parallel relative to the longitudinal axis 150, the thickness of the first blade 134 and the second blade 138 can decrease from a proximal portion 236 of the first blade 134 and second blade 138 to the distal end 104.

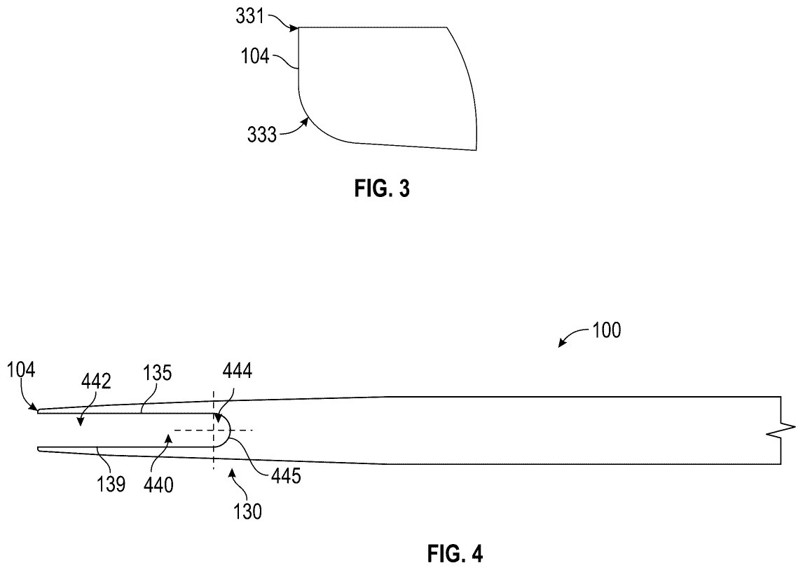

FIG. 3 illustrates a close-up of a cutting edge 331 taken at area 330 illustrated in FIG. 2. In an example, the distal end 104 of at least one of the first blade 134 and second blade 138 can have a cutting edge 331 and an opposing outer face 333. For example, the first inner surface 135 or second inner surface 139, as illustrated in FIG. 1 or 2, can terminate at the cutting edge 331 and the first blade outer surface 133 and the second blade outer surface 137 can terminate at the opposing outer face 333. In an example, the cutting edge 331 can have a beveled profile or a sharp edge profile that can form a sharp point or angle allowing for the cutting edge 331 to penetrate through connections between a bone and an implant. The cutting edge 331 can be a right angle relative to a plane of the distal end 104. The opposing outer face 333 can have a rounded or filet profile. In one example, the opposing outer face 333 can have any profile suitable for the specified purpose. For example, the opposing outer face 333 can have a chamfered or opposing beveled profile. The form of the opposing outer face 333 profile can allow the opposing outer face 333 to pass easily though surrounding tissue.

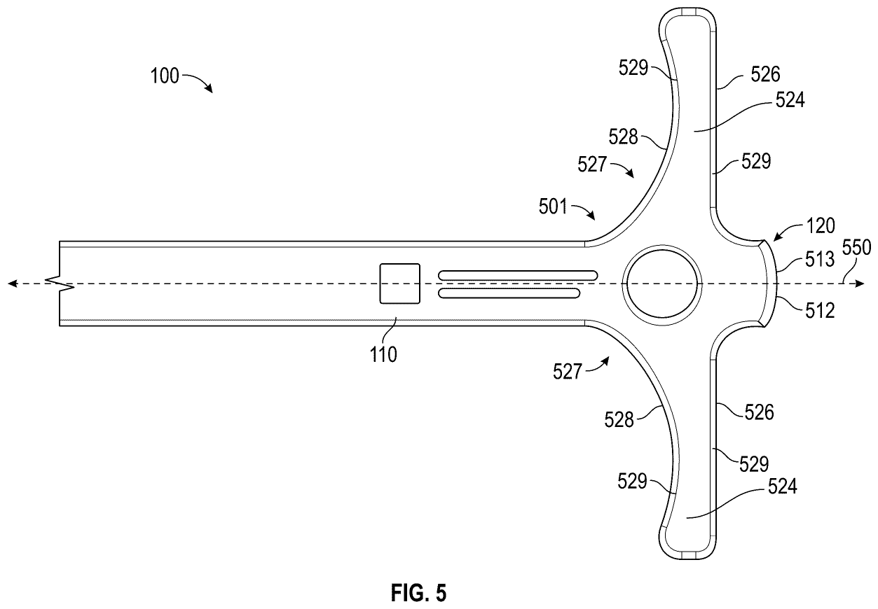

FIG. 4 illustrates a slot 440 (e.g., void, gap, space, or the like) between the first inner surface 135 and the second inner surface 139. The slot 440 can be a space that extends longitudinally and can separate the first inner surface 135 and the second inner surface 139. For example, the slot 440 can extend from a closed proximal end 444 longitudinally spaced from a distal opening 442 of the osteotome 100. The slot 440 can terminate at the closed proximal end 444 spaced a specified distance removed from the distal opening 442. The length of the slot 440 can be specified, for example, by the maximum length the implant extends into a bone or tissue. In an example, the length of the slot 440 can correspond to the maximum length the implant. For example, the slot 440 can be between approximately 10 and approximately 40 millimeters from the distal opening 442 to the closed proximal end 444. In another example, the slot 440 can be between approximately 15 and approximately 30 millimeters from the distal opening 442 to the closed proximal end 444. In another example, the slot 440 can be between approximately 20 and approximately 25 millimeters from the distal opening 442 to the closed proximal end 444.

In one example, the closed proximal end 444 can have a rounded profile 445, such as a “U” shaped profile. The rounded profile 445, in some examples, can reduce stress during use of the working end 130. In some examples, the radius of the rounded profile can be between approximately 0.05 and approximately 2 millimeters. In another example, the radius profile can be between approximately 0.1 and approximately 1 millimeters. In another example, the radius profile can be between approximately 0.05 and approximately 0.5 millimeters. The closed proximal end 444 can also have a partially rounded profile, for example rounded corners. The closed proximal end 444 can also have other profiles such as rectangular or other angular profile. The first inner surface 135 and the second inner surface 139 can extend longitudinally towards the distal end 104 from the closed proximal end 444, thereby forming the slot 440 with opposing faces.

The first inner surface 135 and the second inner surface 139, with the slot 440 disposed between, can form an area that fits around opposing sides of an arm, or other portion or segment, of an implant. For instance, the slot 440 can be formed to accommodate an arm of an implant, such as a shoulder implant. For example, the lateral distance between the first inner surface 135 and the second inner surface 139 can be between about 1 millimeter and about 10 millimeters. In another example, the distance between the first inner surface 135 and the second inner surface 139 can be between about 2 millimeters and about 5 millimeters. In some examples, the lateral distance between the first inner surface 135 and the second inner surface 139 can be substantially the same along the length of the slot 440.

The slot 440 can have a distal opening 442 at the distal end 104 of the working end 130. In one example, the distal opening 442 can be formed having the cutting edge 331, as illustrated in FIG. 3, at the distal end 104 of the first inner surface 135 and the second inner surface 139. For example, the distal end 104 of the first inner surface 135 and the second inner surface 139 can face each other. The space between the distal end 104 of the first inner surface 135 and the second inner surface 139 can be the same or similar distance as the distance between the first inner surface 135 and the second inner surface 139. The space between the distal end 104 of the first inner surface 135 and the second inner surface 139 can be a wider or narrower distance as compared to the distance between the first inner surface 135 and the second inner surface 139, as specified by the purpose of the osteotome 100. For example, the space between the distal end 104 of the first inner surface 135 and the second inner surface 139 can be formed to fit on opposing sides of an arm or other portion of an implant. The distal end 104 at or proximate to the distal opening 442 can include the cutting edge 331, as illustrated in FIG. 3, of each of the first inner surface 135 and the second inner surface 139 can be formed to separate tissue, such as bone, that can be connected to the implant.

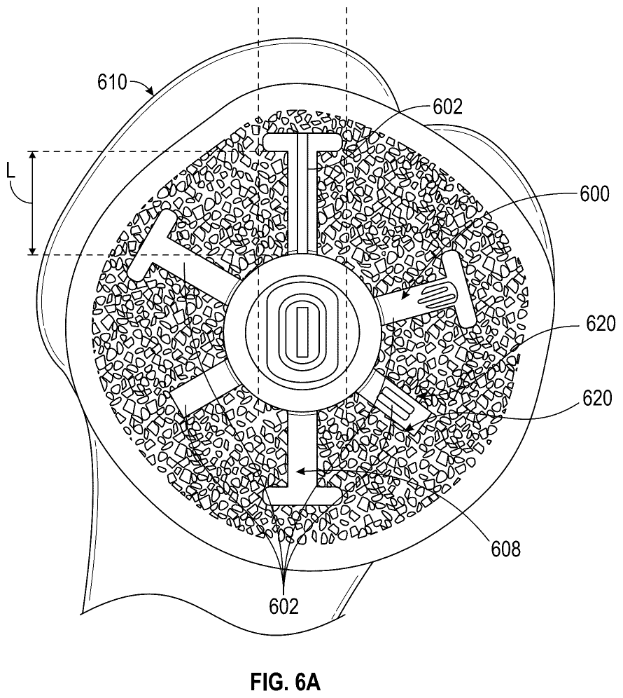

Illustrated in FIG. 5 is a view of an example handle 120 of the osteotome 100 at the proximal end 102 of the osteotome 100. The handle 120 can be formed, molded or otherwise assembled to be coupled with the elongate arm 110. Optionally, the handle 120 can have one continuous arm extending along a proximal portion 501 of the elongate arm 110. In another example, the handle 120 can have two arms 524 extending laterally and away from the elongate arm 110. For example, each of the two arms 524 can extend an equal distance away from the longitudinal axis 550 of the elongate arm 110. The two arms 524 can extend in the same direction as the thickness direction of the first blade 134 and second blade 138, respectively as illustrated in FIGS. 2 and 3.

Each of the two arms 524 can have a proximal or upper surface 526 and an opposing distal or lower surface 528. The proximal or upper surface 526 of each of the two arms 524 can extend away from the longitudinal axis 550 and, in one example, substantially transverse to the longitudinal axis 550. The angle of the proximal or upper surface 526 relative to the longitudinal axis 550 can be an obtuse or acute angle relative to the longitudinal axis 550, as dictated by the purpose. For example, the proximal or upper surface 526 of the handle 120 can be planar and at a substantially right angle forming a striking surface for a mallet or other surgical implement. In an example, the upper surface 526 can be a striking surface, or a surface that can be designed to receive an application of downward force from a separate tool, such as a mallet.

In an example, an extension 512 can project from an area between the upper surface 526 of each of the two arms 524. The extension 512 can be an intermediate striking surface such as a knob or protrusion that can be designed to receive an external downward force. For example, the extension 512 can be collinear with the elongate arm 110. In an example, the longitudinal axis 550 can extend through the elongate arm 110 and the extension 512 with each of the proximal or upper surfaces 526 extending therefrom. The extension 512 can have a planar upper or most proximal surface 513. In one example, the planar upper surface 513 or most proximal surface can be transverse to the longitudinal axis 550. The planar upper surface 513 can be formed to be a striking surface, or surface that receives an external, laterally applied force.

Opposing the upper surface 526 can be a lower surface 528 that can be formed to be gripped or held by a medical professional. In one example, the opposing distal or lower surface 528 can have an arcuate profile 527. The arcuate profile 527 can be an ergonomic form providing a gripping surface for a medical professional to use the osteotome 100. For example, the opposing distal or lower surface 528 of each of the two arms 524 can be similarly curved to allow the fingers of a medical professional to easily grip the handle 120. The opposing distal or lower surface 528 can have a different arcuate profile 527 associated with each of the two arms 524. The design of the lower surface 528 can depend on the use of the osteotome 100, such as how the handle 120 is held.

Optionally, the handle 120 can have edges 529 that can have a beveled or filet profile. The edges 529 with a beveled or filet profile can provide a more comfortable user experience for the medical professional. In some examples, the edges 529 can be sharp edges formed to simplify manufacturing. The handle 120 can be configured or formed in any design suitable for a medical professional to grip, hold or otherwise engage with the osteotome 100 while applying a force, such as a repeated force, to the proximal end 102.

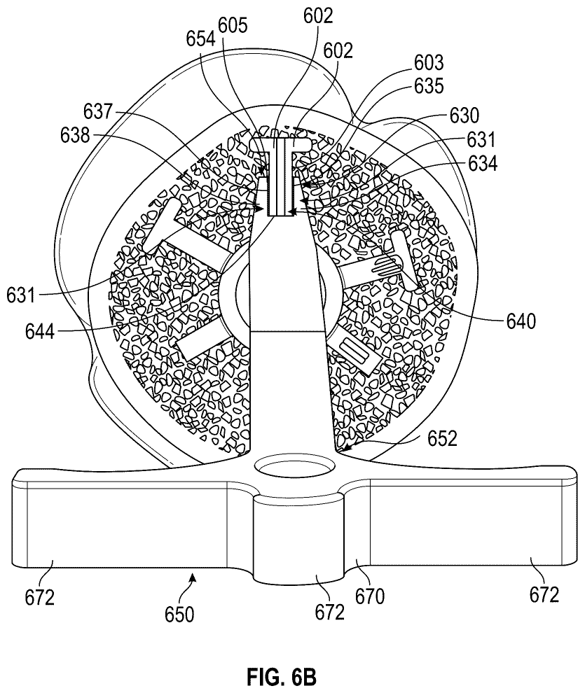

Illustrated in FIG. 6A is an example of an implant 600 previously inserted into, for example, a bone 610. Some surgical procedures can be benefited from the removal of an implant 600. In such procedures, connections 620 can exist between the bone 610 and the implant 600. In some instances, the connections 620 cannot be easily broken or fractured and a medical professional can benefit from using a surgical instrument to separate, break or fracture the connections 620 between the bone 610 and the implant 600.

In an example method using the osteotome 650 (as illustrated in FIG. 6B), the medical professional can shorten or minimize the time and effort of work performed in separating the tissue or bone from the implant 600, as compared to those methods using a traditional osteotome. The minimization of time and effort can be, at least in part, because the profile of osteotome 650 having a first blade 634 and second blade 638. For instance, the first blade 634 and the second blade 638 can be designed for insertion into a bone on opposing sides of a portion or segment of the implant 600. In an example method of using the osteotome 650, the medical professional would not have to use a single-bladed osteotome on one side to separate the bone from the implant and then repeat the activity on the other side. In an example using the osteotome 650, the connections 620 between the bone and a location on each side of a portion, such as an arm, of the implant can be fractured or separated in a single pass.

In an example of a method for removal of an implant, an osteotome 650 (similar to osteotome 100 discussed related to FIGS. 1-5), as illustrated in FIG. 6B, can be implemented as part of an implant removal system. For example, removal of the implant 600 can include a method of inserting the osteotome 650, into the bone 610 or tissue in areas where the bone 610 or tissue is united (e.g., joined, coupled, bound or the like) with the implant 600. In an example, the osteotome 650 can be inserted into the tissue or bone 610 around the implant 600.

In a method of inserting the osteotome 650 into the bone, a distal end 654 of the osteotome 650 can be aligned proximate to at least a portion or segment of the implant 600. For example, a first blade 634 and a second blade 638 of the osteotome 650 can be aligned proximate to at least a portion of the implant 600 or abutting at least a portion of the implant 600. The first blade 634 can have a first inner surface 635 and the second blade 638 can have a second inner surface 637 facing the first inner surface 635. The first inner surface 635 and the second inner surface 637 each can extend distally and can be aligned on opposing sides of a portion, such as an arm 602 of the implant 600. For example, the first blade 634 can be oriented to abut at least a segment of a first side 603 of the arm 602 and the second blade 638 can be oriented to abut at least a segment of the second side 605 of the arm 602 when the first blade 634 and second blade 638 are inserted into the bone 610.

In an example, after each of the first blade 634 and the second blade 638 have been positioned, such as aligned, on opposing sides of the arm 602, a lateral or downward force can be applied to a proximal end 652 of the osteotome 650. For example, a medical professional can use a mallet to strike the upper surfaces 672 of a handle 670 at the proximal end 652 of the osteotome 650. For instance, the medical professional can use a tool to apply a downward force against the upper surfaces 672 to further insert the osteotome 650 into the bone 610.

In an example using a mallet to apply a force, including a repeated force, such as hammering, can be applied to the proximal end 652 of the osteotome 650. A corresponding lateral force, such as a mallet strike against the upper surface 672 of the handle 670, can be transferred from the proximal end 652 of the osteotome 650 to the distal end 654. For example, the force can be transferred to at least one of the distal end 654 of the first blade 634 or the second blade 638. The force, in another example, can be transferred to the distal end 654 of both the first blade 634 and the second blade 638. The force can also be transferred a substantially equal amount to the first blade 634 and the second blade 638 when applying repeated force to the proximal end 652 of the osteotome 650.

The application of force can result in the distal end 654 of the first blade 634 being inserted in concert (e.g., at the substantially the same time, within milliseconds of each other, within one second) with the second blade 638 into the bone or tissue. As further force is applied to the distal end 654 of one or more of the first blade 634 or the second blade 638, the first blade 634 and second blade 638 can each be further inserted into the bone 610 on opposing sides of the arm 602. With repeated application of force to the proximal end 652, the distal end 654 can be further inserted into the bone 610. The first blade 634 and second blade 638 can be inserted into the bone 610 a specified distance. For example, the first blade 634 and second blade 638 can be inserted until a proximal end 644 of a slot 640 extending between the first blade 634 and second blade 638 contacts an exposed surface 608 of the implant 600. In another example, a portion of the length of the first blade 634 and second blade 638 can be inserted on opposing sides of the arm 602 and into the bone 610. Insertion of the first blade 634 and second blade 638 can result in fracturing the connections 620 between the bone 610 and the implant 600 on both sides of the arm 602 of the implant 600 without reinsertion of the osteotome 650 on an opposing side of the arm 602.

After inserting the first blade 634 and second blade 638 the specified amount, the medical professional can withdraw, or can remove the first blade 634 and the second blade 638 from the bone 610. For example, the first blade 634 and the second blade 638 can be withdrawn concurrently. In another example, the osteotome 650 can be moved with small movements, such as by wiggling, one of the first blade 634 or the second blade 638, to release the first blade 634 and the second blade 638 from the bone 610. In such a situation, the first blade 634 or the second blade 638 can be removed in sequence.

In an example, sides 631 of the first blade 634 and the second blade 638 may not be as wide as the length L of the arm 602. After removal of the first blade 634 and the second blade 638, the blade 630 can be repeatedly inserted along the length L of the arm 602. The method of inserting the osteotome 650 along the length L can be repeated along each segment or portion of the implant 600. For instance, the method of inserting the osteotome 650 can be repeated along each of the arms 602 until the implant 600 is disengaged from the bone 610 and can be removed.

### VARIOUS NOTES AND ASPECTS

Aspect 1 can include surgical instrument configured to be inserted into a bone comprising: an elongate arm having a proximal end, a central body, and a distal end; a handle having a force application surface, the handle coupled to the proximal end of the elongate arm; and a working end proximate to the distal end of the elongate arm, the working end including: a first blade having a first outer surface and an inwardly facing first surface and a second blade having a second outer surface and an inwardly facing second surface; wherein the inwardly facing first surface faces the inwardly facing second surface; wherein the elongate arm continuously tapers from the central body to a distal end of each of the first blade and the second blade; wherein the distal end of each of the first blade and the second blade are configured to be inserted into the bone; and a slot extending longitudinally between the inwardly facing first surface and the inwardly facing second surface; wherein the slot is open at the distal end and terminates in a closed configuration a specified distance removed from the distal end.

Aspect 2 can include, or can optionally be combined with the subject matter of Aspect 1, to optionally include a longitudinal axis of the elongate arm centered between the inwardly facing first surface and the inwardly facing second surface.

Aspect 3 can include, or can optionally be combined with the subject matter of one or any combination of Aspects 1 or 2 to optionally include the handle includes at least two arms, each arm extending transverse to a longitudinal axis of the elongate arm.

Aspect 4 can include, or can optionally be combined with the subject matter of one or any combination of Aspects 1-3 to optionally include the first outer surface of the first blade and the second outer surface of the second blade each taper towards the distal end.

Aspect 5 can include, or can optionally be combined with the subject matter of one or any combination of Aspects 1-4 to optionally include the first outer surface has a first taper at a first angle and a second taper extending from the first taper at a second angle, the second taper terminating at the distal end of the first blade; and wherein the second outer surface has a first taper at a first angle and a second taper extending from the first taper at a second angle, the second taper terminating at the distal end of the second blade.

Aspect 6 can include, or can optionally be combined with the subject matter of one or any combination of Aspects 1-5 to optionally include the handle has a first planar proximal surface and a second concave surface longitudinally opposite to the first planar proximal surface.

Aspect 7 can include, or can optionally be combined with the subject matter of one or any combination of Aspects 1-6 to optionally include the closed configuration of the slot has a u-shape profile.

Aspect 8 can include, or can optionally be combined with the subject matter of one or any combination of Aspects 1-7 to optionally include the first blade and the second blade are symmetrical about a longitudinal axis of the elongate arm.

Aspect 9 can include, a method of inserting a surgical instrument having a proximal end, a central body, a distal end, and a first blade with a first inwardly facing surface extending distally from the central body and a second, opposing blade with a second inwardly facing surface extending distally from the central body, the first blade and second blade configured to be inserted into a bone, the method comprising: aligning a distal end of the first blade and a distal end of a second blade on opposing sides of an object implanted into the bone; applying a force to the proximal end of the surgical instrument; wherein the force is transferred from the proximal end of the surgical instrument to the distal end of the first blade and the distal end of the second blade; inserting the distal end of the first blade in concert with the distal end of the second blade into the bone, on opposing sides of the object with at least one application of the force; ceasing the application of the force when the first blade and the second blade extend a specified distance into the bone; and concurrently withdrawing the first blade and the second blade from the bone.

Aspect 10 can include, or can optionally be combined with the subject matter of Aspect 9, to optionally include fracturing a bond between surfaces of the object and the bone by inserting the first blade and the second blade into the bone.

Aspect 11 can include, or can optionally be combined with the subject matter of one or any combination of Aspects 9 or 10 to optionally include aligning the distal end of the first blade and the distal end of the second blade are aligned in contact with at least one side of a medical implant.

Aspect 12 can include, or can optionally be combined with the subject matter of one or any combination of Aspects 9-11 to optionally include transferring a substantially equal force to the first blade and the second blade when applying a repeated force to the proximal end of the surgical instrument.

Aspect 13 can include, or can optionally be combined with the subject matter of one or any combination of Aspects 9-12 inserting the distal end of the first blade and the distal end of the second blade includes insertion on opposing sides and in contact with at least a portion of an implant.

Aspect 14 can include, or can optionally be combined with the subject matter of one or any combination of Aspects 9-13 orienting the first blade to abut at least a segment of a first side of the opposing sides of the object and orienting the second blade to abut at least a segment of a second side of the opposing sides of the object when the first blade and second blade are inserted into the bone.

Aspect 15 can include, or can optionally be combined with the subject matter of one or any combination of Aspects 9-14 ceasing application of force when the proximal end of the first blade and the proximal end of the second blade contact an exposed surface of either the object or an exposed surface of the bone. Aspect 16 can include a surgical implant removal system comprising: an implant configured to be implanted in a bone; wherein the implant is configured to be united with the bone; and a surgical instrument configured to be inserted into a bone comprising: an elongate arm having a proximal end, a central body, and a distal end; a handle having a force application surface, the handle coupled to the proximal end of the elongate arm; and a working end proximate to the distal end of the elongate arm, the working end including: a first blade having a first outer surface and an inwardly facing first surface and a second blade having a second outer surface and an inwardly facing second surface; wherein the inwardly facing first surface faces the inwardly facing second surface; wherein the elongate arm continuously tapers from the central body to a distal end of each of the first blade and the second blade; wherein the distal end of each of the first blade and the second blade are configured to be inserted into the bone; and a slot extending longitudinally between the first surface and the second surface; wherein the slot is open at the distal end and terminates in a closed configuration a specified distance removed from the distal end.

Aspect 17 can include, or can optionally be combined with the subject matter of Aspect 16, to optionally include handle includes at least two arms, each arm extending transverse to a longitudinal axis of the elongate arm.

Aspect 18 can include, or can optionally be combined with the subject matter of one or any combination of Aspects 16 or 17 to optionally include wherein the surgical instrument is configured to fracture connections between surfaces of the implant and the bone.

Aspect 19 can include, or can optionally be combined with the subject matter of one or any combination of Aspects 16-18 to optionally include the handle or the proximal end is configured to receive a repeated force; wherein the handle has a first planar proximal surface and a second concave surface longitudinally opposite to the first planar proximal surface.

Aspect 20 can include, or can optionally be combined with the subject matter of one or any combination of Aspects 16-19 to optionally include the first outer surface has a first taper at a first angle and a second taper extending from the first taper at a second angle, the second taper terminating at the distal end of the first blade; and wherein the second outer surface has a first taper at a first angle and a second taper extending from the first taper at a second angle, the second taper terminating at the distal end of the second blade.

Each of these non-limiting aspects can stand on its own, or can be combined in various permutations or combinations with one or more of the other aspects.

The above description includes references to the accompanying drawings, which form a part of the detailed description. The drawings show, by way of illustration, specific embodiments in which the invention can be practiced. These embodiments are also referred to herein as “aspects” or “examples.” Such aspects or example can include elements in addition to those shown or described. However, the present inventors also contemplate aspects or examples in which only those elements shown or described are provided. Moreover, the present inventors also contemplate aspects or examples using any combination or permutation of those elements shown or described (or one or more features thereof), either with respect to a particular aspects or examples (or one or more features thereof), or with respect to other Aspects (or one or more features thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

The above description is intended to be illustrative, and not restrictive. For example, the above-described aspects or examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as aspects, examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A surgical instrument configured to be inserted into a bone comprising:
an elongate arm having a proximal end, a central body, and a distal end;
a handle having a force application surface, the handle coupled to the proximal end of the elongate arm; and
a working end proximate to the distal end of the elongate arm, the working end including:
a first blade having a first outer surface and an inwardly facing first surface and a second blade having a second outer surface and an inwardly facing second surface;
wherein the inwardly facing first surface faces the inwardly facing second surface;
wherein the elongate arm continuously tapers from the central body to a distal end of each of the first blade and the second blade;
wherein the distal end of each of the first blade and the second blade are configured to be inserted into the bone; and
a slot extending longitudinally between the inwardly facing first surface and the inwardly facing second surface;
wherein the slot is open at the distal end and terminates in a closed configuration a specified distance removed from the distal end.

2. The surgical instrument of claim 1, including a longitudinal axis of the elongate arm centered between the inwardly facing first surface and the inwardly facing second surface.

3. The surgical instrument of claim 1, wherein the handle includes at least two arms, each arm extending transverse to a longitudinal axis of the elongate arm.

4. The surgical instrument of claim 1, wherein the first outer surface of the first blade and the second outer surface of the second blade each taper towards the distal end.

5. The surgical instrument of claim 1, wherein the first outer surface has a first taper at a first angle and a second taper extending from the first taper at a second angle, the second taper terminating at the distal end of the first blade; and
wherein the second outer surface has a first taper at a first angle and a second taper extending from the first taper at a second angle, the second taper terminating at the distal end of the second blade.

6. The surgical instrument of claim 1, wherein the handle has a first planar proximal surface and a second concave surface longitudinally opposite to the first planar proximal surface.

7. The surgical instrument of claim 1, wherein the closed configuration of the slot has a u-shape profile.

8. The surgical instrument of claim 1, wherein the first blade and the second blade are symmetrical about a longitudinal axis of the elongate arm.

9. A method of inserting a surgical instrument having a proximal end, a central body, a distal end, and a first blade with an inwardly first facing surface extending distally from the central body and a second, opposing blade with an inwardly second facing surface extending distally from the central body, the first blade and second blade configured to be inserted into a bone, the method comprising:
aligning a distal end of the first blade and a distal end of a second blade on opposing sides of an object implanted into the bone;
applying a force to the proximal end of the surgical instrument;
wherein the force is transferred from the proximal end of the surgical instrument to the distal end of the first blade and the distal end of the second blade;
inserting the distal end of the first blade in concert with the distal end of the second blade into the bone, on opposing sides of the object with at least one application of the force;
ceasing the application of the force when the first blade and the second blade extend a specified distance into the bone; and
concurrently withdrawing the first blade and the second blade from the bone.

10. The method of claim 9, including fracturing a bond between surfaces of the object and the bone by inserting the first blade and the second blade into the bone.

11. The method of claim 9, wherein aligning the distal end of the first blade and the distal end of the second blade are aligned in contact with at least one side of a medical implant.

12. The method of claim 9, including transferring a substantially equal force to the first blade and the second blade when applying a repeated force to the proximal end of the surgical instrument.

13. The method of claim 9, wherein inserting the distal end of the first blade and the distal end of the second blade includes insertion on opposing sides and in contact with at least a portion of an implant.

14. The method of claim 9, including orienting the first blade to abut at least a segment of a first side of the opposing sides of the object and orienting the second blade to abut at least a segment of a second side of the opposing sides of the object when the first blade and second blade are inserted into the bone.

15. The method of claim 9, including ceasing application of force when the proximal end of the first blade and the proximal end of the second blade contact an exposed surface of either the object or an exposed surface of the bone.

16. A surgical implant removal system comprising:
an implant configured to be implanted in a bone;
wherein the implant is configured to be united with the bone; and
a surgical instrument configured to be inserted into a bone comprising:
an elongate arm having a proximal end, a central body, and a distal end;
a handle having a force application surface, the handle coupled to the proximal end of the elongate arm; and
a working end proximate to the distal end of the elongate arm, the working end including:
a first blade having a first outer surface and an inwardly facing first surface and a second blade having a second outer surface and an inwardly facing second surface;
wherein the inwardly facing first surface faces the inwardly facing second surface;
wherein the elongate arm continuously tapers from the central body to a distal end of each of the first blade and the second blade;
wherein the distal end of each of the first blade and the second blade are configured to be inserted into the bone; and
a slot extending longitudinally between the inwardly facing first surface and the inwardly facing second surface;
wherein the slot is open at the distal end and terminates in a closed configuration a specified distance removed from the distal end.

17. The surgical implant removal system of claim 16, wherein the handle includes at least two arms, each arm extending transverse to a longitudinal axis of the elongate arm.

18. The surgical implant removal system of claim 16, wherein the surgical instrument is configured to fracture connections between surfaces of the implant and the bone.

19. The surgical implant removal system of claim 16, wherein the handle or the proximal end is configured to receive a repeated force;
wherein the handle has a first planar proximal surface and a second concave surface longitudinally opposite to the first planar proximal surface.

20. The surgical implant removal system of claim 16, wherein the first outer surface has a first taper at a first angle and a second taper extending from the first taper at a second angle, the second taper terminating at the distal end of the first blade; and
wherein the second outer surface has a first taper at a first angle and a second taper extending from the first taper at a second angle, the second taper terminating at the distal end of the second blade.
